(19) United States
(12) Patent Application Publication
(10) Pub. No.: US 2009/0187017 A1
(43) Pub. Date: Jul. 23, 2009

(54) PROCESS FOR PREPARING ENANTIOMERICALLY ENRICHED BETA-AMINO ACID DERIVATIVES

(75) Inventors: Albrecht Berkessel, Erftstadt (DE); Felix Cleemann, Hürth (DE); Santanu Mukherjee, Köln (DE)

Correspondence Address: OBLON, SPIVAK, MCCLELLAND MAIER & NEUSTADT, P.C., 1940 DUKE STREET, ALEXANDRIA, VA, 22314, US

(73) Assignee: Evonik Degussa GmbH, Essen (DE)

(21) Appl. No.: 12/064,194

(22) PCT Filed: Jul. 25, 2006
(86) PCT No.: PCT/EP06/64615

§ 371 Date: Aug. 4, 2008

(30) Foreign Application Priority Data
Aug. 25, 2005 (DE) 102005040155.4

(51) Int. Cl. C07D 265/06; C07C 233/87

(52) U.S. Cl. 544/97; 560/41

(57) ABSTRACT

The present invention relates to a process for the organo-catalysed kinetic racemate resolution of compounds of the general formula (II). It is thus possible through the action of catalytic amounts of enantiomerically enriched compounds of the general formula (Ia) or (Ib) to obtain on the one hand enantiomerically enriched optionally N-acylated β-amino acid esters and on the other hand enantiomerically enriched 4,5-dihydrooxazin-6-ones (oxazinones). The corresponding enantiomerically enriched β-amino acids can be formed from both easily separable classes of compounds by simple hydrolysis.

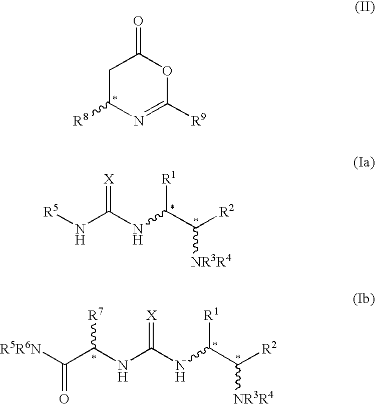

The present invention relates to a process for preparing enantiomerically enriched, optionally N-acylated, β-amino acid esters and optionally N-acylated β-amino acids and enantiomerically enriched 4,5-dihydrooxazin-6-ones (oxazinones).

Optically active β-amino carboxylic acids (β-amino acids) occur in natural products such as alkaloids and antibiotics. Isolation thereof is therefore of increasing interest, not least because of the increasing importance in the area of intermediates in the preparation of pharmaceuticals (see inter alia: E. Juaristi, H. Lopez-Ruiz, Curr. Med. Chem. 1999, 6, 983-1004). Both the free form of optically active β-amino carboxylic acids and the derivatives thereof show interesting pharmacological effects and can also be employed in the synthesis of modified peptides.

A large number of methods is available in principle for preparing enantiomerically enriched β-amino acids and derivatives thereof, but the number of industrially applicable syntheses is limited.

For example, the preparation of β-aminocarboxylic acids can be effected with the aid of classical racemate resolution via diastereomeric pairs of salts (proposed route in: H. Boesch et al., Org. Proc. Res. Developm. 2001, 5, 23-27) or with the aid of a diastereoselective synthesis via the diastereoselective addition of optically active lithium phenylethylamide (A. F. Abdel-Magid, J. H. Cohen, C. A. Maryanoff, Curr. Med. Chem. 1999, 6, 955-970). The latter method is regarded as intensively researched and is preferably applied despite numerous disadvantages occurring therewith. It is regarded as disadvantageous on the one hand that stoichiometric amounts of a chiral reagent are required, which represents a large disadvantage compared with catalytic asymmetric methods. The agent which is required in stoichiometric amounts can moreover not be recycled again, which represents a further disadvantage. In addition, costly auxiliaries which are moreover problematic in terms of industrial safety, such as, for example, n-butyllithium, are required to activate the stoichiometric reagent by deprotonation. In addition, it is important to carry out the reaction at low temperatures of about −70° C. for sufficiently high stereoselectivity, making great demands on the reactor material and many additional costs and high energy consumption.

Alternative solutions are biocatalytic approaches which have proved to be suitable in particular also for industrial applications. Preferred enzyme-based syntheses are in this connection the penicillin G amidase-catalysed racemate resolution by hydrolysis of N-phenylacetylated β-amino acids (V. A. Soloshonok, V. K. Svedas, V. P. Kukhar, A. G. Kirilenko, A. V. Rybakova, V. A. Solodenko, N. A. Fokina, O. V. Kogut, I. Y. Galaev, E. V. Kozlova, I. P. Shishkina and S. V. Galushko, Synlett 1993, 339-341; V. A. Soloshonok, A. G. Kirilenko, N. A. Fokina, I. P. Shishkina, S. V. Galushko, V. P. Kukhar, V. K. Svedas and E. V. Kozlova, Tetrahedron: Asymmetry 1994, 5, 1119-1126; V. A. Soloshonok, N. A. Fokina, A. V. Rybakova, I. P. Shishkina, S. V. Galushko, A. E. Sochorinsky, V. P. Kukhar, M. V. Savchenko and V. K. Svedas, Tetrahedron: Asymmetry 1995, 6, 1601-1610; G. Cardillo, A. Tolomelli and C. Tomasini, Eur. J. Org. Chem. 1999, 155-161) and the lipase-catalysed racemate resolution by hydrolysis of N-unprotected or N-protected β-amino acid esters (S. G. Cohen, S. Y. Weinstein, J. Am. Chem. Soc. 1964, 86, 725; M. Prashad, D. Har, O. Repic, T. J. Blacklock, P. Giannousis, Tetrahedron: Asymmetry 1998, 9, 2133-2136; S. Katayama, N. Ae, R. Nagata, Tetrahedron: Asymmetry 1998, 9, 4295-4299; S. J. Faulconbridge, K. E. Holt, L. G. Sevillano, C. J. Lock, P. D. Tiffin, N. Tremayne, S. Winter, Tetrahedron Lett. 2000, 41, 2679-2681; U.S. Pat. No. 6,869,781). The concept of lipase-catalysed racemate resolution by ester hydrolysis of β-amino acid esters has been applied industrially in the production of enantiomer pure aryl-substituted β-amino acids, it being possible to achieve high enantiomeric excesses of >99% ee for the isolated products. However, despite the high efficiency of the synthesis, the substrate spectrum is limited owing to the specificity of the enzymes: whereas aromatic substrates are tolerated very well, it proved to be difficult to obtain aliphatic representatives. Thus, for example, it is not possible to obtain the sterically demanding, optically active β-neopentylglycine sufficiently well in this way.

It is therefore an object of the present invention to provide a further process for preparing β-amino acids. It was intended that such a process be superior to prior art processes in relation to ecological and economic aspects. It was particularly intended that the present process permit linear and branched-substituted β-amino acids also to be prepared on the industrial scale.

These objects, and further objects which are not specified in detail but are obvious from the prior art are achieved by a process according to the features of Claim 1. Further preferred embodiments of the process according to the invention are protected in the subclaims dependent on Claim 1.

The stated objects are achieved in a simple but extremely advantageous manner in a process for preparing enantiomerically enriched compounds selected from the group consisting of N-acylated β-amino acid esters, β-amino acid esters, N-acylated β-amino acids, β-amino acids and 4,5-dihydrooxazin-6-ones by kinetic racemate resolution starting from, for example, the 4,5-dihydrooxazin-6-one formed from the corresponding racemic N-acylated β-amino acid, and reacting it with catalytic amounts of an enantiomerically enriched compound of the general formula (Ia) or (Ib),

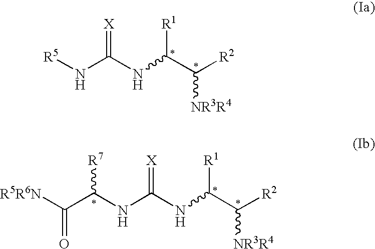

in which
* represents chiral centres,

### X may be O or S or NR¹R² or NHR¹,

R¹, R², R³, R⁴, R⁵, R⁶, R⁷ are independently of one another (C₁-C₈)-alkyl, (C₁-C₈)-alkoxy, HO— (C₁-C₈)-alkyl, (C₂-C₈)-alkoxyalkyl, (C₆-C₁₈)-aryl, (C₇-C₁₉)-aralkyl, (C₃-C₁₈)-hetero-aryl, (C₄-C₁₉)-heteroaralkyl, (C₁-C₈)-alkyl-(C₆-C₁₈)-aryl, (C₁-C₈)-alkyl-(C₃-C₁₈)-heteroaryl, (C₃-C₈)-cycloalkyl, (C₁-C₈)-alkyl-(C₃-C₈)-cycloalkyl, (C₃-C₈)cycloalkyl-(C₁-C₈)-alkyl, and
R¹ and R² and/or R² and R³ may be connected together via a (C₃-C₅)-alkylene bridge, in the presence of a nucleophile. The process is surprisingly suitable, by comparison with previously disclosed catalysis systems, for preparing both aromatic and aliphatic-substituted derivatives of β-amino acids.

The following reaction scheme illustrates the reaction according to the invention:

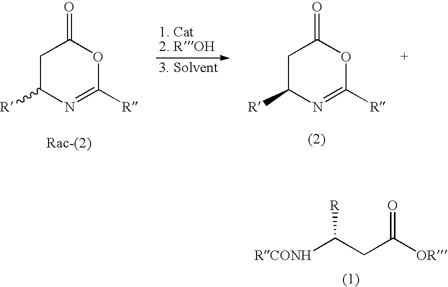

Both enantiomerically enriched compounds ((1) and (2)) can subsequently be converted as decided by the skilled person directly—e.g. by hydrolysis—into the correspondingly optically active β-amino acids.

Preferred catalysts are structures of the following type:

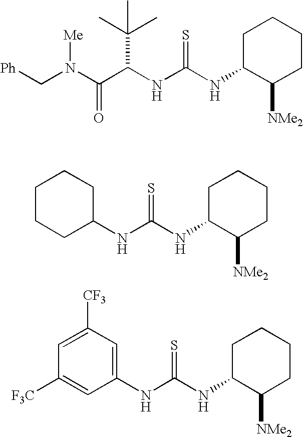

The starting compounds of the oxazinone type employed for the reaction according to the invention can be prepared as decided by the skilled person (C. Drey, E. Mtetwa, J. Chem. Soc., Perkin Trans. 1 1982, 587-1592). An advantageous preparation starts from the racemic β-amino acid, which is acylated on the nitrogen atom in a Schotten-Baumann reaction and is then ring-closed to the oxazinone under dehydrating conditions, for example through the action of organic or inorganic acid anhydrides.

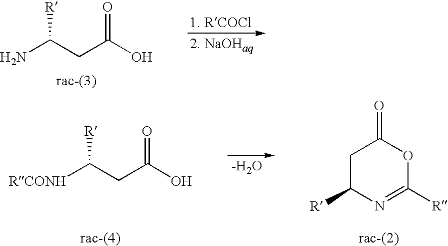

The reaction can be carried out with 2,4-, 2,4,5- or 2,5-substituted 4,5-dihydrooxazinones.

Preferably employed for the reaction according to the invention are 2,4-disubstituted 4,5-dihydrooxazin-6-ones of the general formula (II),

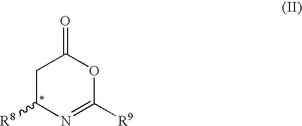

in which
* represent chiral centres
R⁸, R⁹ (C₁-C₁₈)-alkyl, (C₁-C₈)-alkoxy, HO—(C₁-C₈)-alkyl, (C₂-C₈)-alkoxyalkyl, (C₆-C₁₈)-aryl, (C₇-C₁₉)-aralkyl, (C₃-C₁₈)-heteroaryl, (C₄-C₁₉)-heteroaralkyl, (C₁-C₈)-alkyl-(C₆-C₁₈)-aryl, (C₁-C₈)-alkyl-(C₃-C₁₈)-heteroaryl, (C₃-C₈)-cycloalkyl, (C₁-C₈)-alkyl-(C₃-C₈)-cycloalkyl, (C₃-C₈)-cyclo-alkyl-(C₁-C₈)-alkyl. It is very particularly preferred to employ compounds of the general formula (II) in which R⁹ is a (C₆-C₁₈)-aryl radical, in particular a phenyl radical. It is also very particularly preferred to use for the racemate resolution those compounds of the general formula (II) in which the radical R⁸ is (C₁-C₁₈)-alkyl, in particular a bulky, branched alkyl group having a tertiary C atom and 4-10 C atoms, for example tert-butyl or neopentyl, and is (C₆-C₁₈)-aryl, in particular phenyl.

The reaction according to the invention proceeds in such a way that the stereoselective opening of the employed oxazinone takes place through the action of a nucleophile. Alcohols are preferably employed for this purpose. The alcohols advantageously selected are those which can subsequently be easily eliminated by acidic or basic hydrolysis in order to be able to convert the enantiomerically enriched N-acylated β-amino acid esters which are formed into the desired amino acids in a simple manner. The alcohols therefore preferably employed for the reaction are compounds selected from the group consisting of allyl alcohol, methyl alcohol, ethyl alcohol, phenol, benzyl alcohol, n- or iso-propyl alcohol and n-, tert-, sec- and iso-butanol. The use of allyl alcohol is very particularly preferred in this connection. Besides these alcohols, however, it is also possible to employ water or OH⁻ ions or thiols or amines as nucleophiles. Where water or OH⁻ ions are employed, the N-acylated β-amino acids would be obtained directly. If the intention is to obtain these, water or OH⁻ ions is to be preferred as nucleophile. The pH range in which the reaction can be carried out is to be decided by the skilled person.

The skilled person has a free choice of the amount of catalyst employed for the reaction. The catalysts of the general formula (I) can preferably be employed in the range from 0.01 to 40 mol % based on the substrate for kinetic racemate resolution. A range from 0.5 to 10 mol % is more preferred, and one from 1 to 5 mol % is very particularly preferred.

The temperature which is set for the kinetic racemate resolution can be chosen by the skilled person as desired. The main basis for him in this connection is the fact that the enantiomeric excesses in the products are as high as possible and the reaction rate is not substantially slowed down. It has emerged that the reaction proceeds optimally when a temperature of between about 15° C. and 40° C. is set during the reaction. The preferred range is located at 20° C. and 30° C.

The process according to the invention is carried out in organic solvents which show inert behaviour during the kinetic racemate resolution. They should furthermore be able to dissolve to a sufficient extent the rel. polar compounds. Organic solvents selected from the group of organic aprotic solvents are therefore preferably employed in the subject reaction. It is very particularly preferred to employ toluene, dichloromethane, acetonitrile or mixtures thereof.

Catalysts of the general formula (I) which are in enantiomerically enriched form are employed for the reaction according to the invention. Such catalysts with an enantiomeric enrichment of >80% ee are preferably employed in the kinetic racemate resolution. It is more preferred to employ catalysts with an enantiomeric enrichment of >90% ee, further preferably >95% ee and very particularly preferably >98% ee.

Chosen as example of the kinetic racemate separation is the reaction of rac-4,5-dihydro-2,4-diphenyl-1,3-oxazin-6-one (4) with allyl alcohol (see diag. 1).

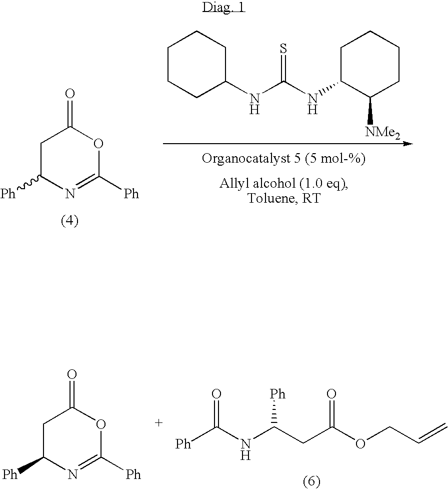

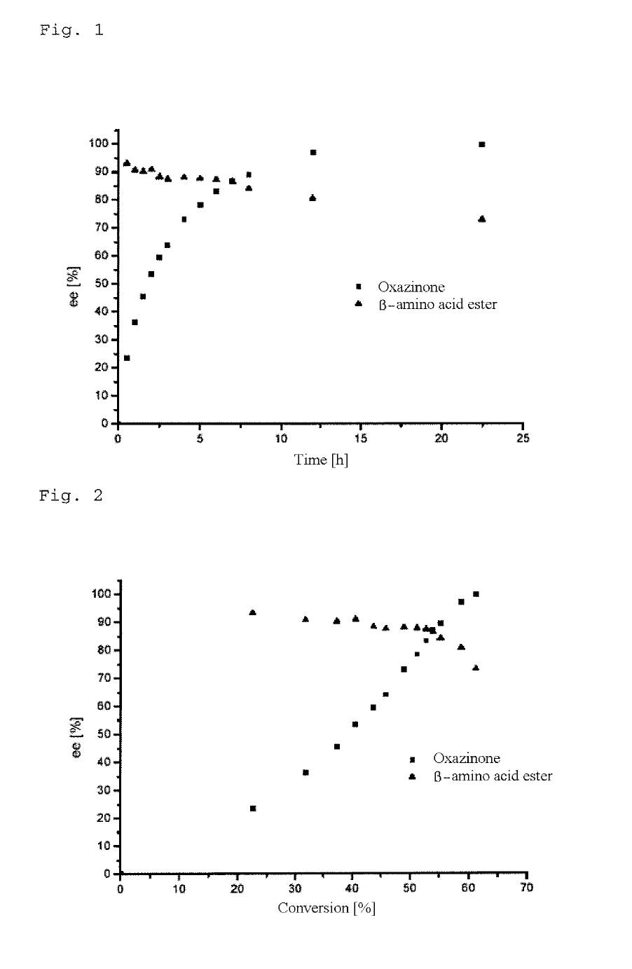

A 0.1 M solution of the oxazinone in abs. toluene was prepared and to this were added 1.0 eq. of allyl alcohol and 0.05 eq. of the bifunctional thiourea catalyst 5. Catalysts of this type can be synthesized in one step by reacting a chiral diamine with isothiocyanates (T. Okino, Y. Hoashi, Y. Takemoto, J. Am. Chem. Soc. 2003, 125, 12672-12673). Samples were taken from the reaction solution at defined times, and the enantiomeric excess of the employed oxazinone, of the resulting β-amino acid ester (6) and the conversion of the reaction were determined by chiral HPLC. The results show the characteristics expected for a kinetic racemate separation and are depicted in graphic form in FIG. 1 and FIG. 2. It emerged that one enantiomer of the racemic substrate reacts preferentially, so that the enantiomeric excess of the oxazinone increases continuously until only one enantiomer remains. The enantiomeric excess of the product declines slowly as conversion advances (FIG. 1 and FIG. 2). Both the remaining oxazinone and the N-benzoyl-β-amino acid ester formed can be converted without loss of enantiomeric purity into the free β-amino acids. It is thus possible in one reaction to obtain both enantiomers of the β-amino acid. It was possible to show in further experiments that the structurally analogous thiourea compounds 7 and 8 are also effective as active and highly enantioselective catalysts of the kinetic racemate separation of oxazinone 4. Tab. 1 indicates the conversions obtained.

TAB. 1
Catalysis results using various
bifunctional catalysts.

| | | % | % |
|---|---|---|---|
| | % | ee | ee |
| Catalyst | conversion | (4) | (6) |
| 5 | 61 | >99 | 73 |
| 7 | 60 | >99 | 75 |
| 8 | 57 | 99 | 86 |

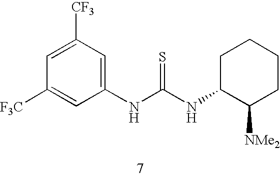

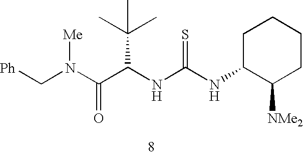

In summary, it can be said that the described process represents a highly efficient and selective route to enantiomer pure β-amino acids. It is to be expected that this method will be applicable also to the synthesis of many other β-amino acid derivatives. It is a great benefit in this connection that the catalysts used have a modular structure and thus can easily be adapted to new substrates.

(C₁-C₈)-Alkyl radicals are to be regarded as being methyl, ethyl, n-propyl, isopropyl, n-butyl, isobutyl, sec-butyl, tert-butyl, pentyl, hexyl, heptyl or octyl including all their bond isomers.

A (C₁-C₁₈)-alkyl radical is within the scope of the definition according to the invention a corresponding (C₁-C₈)-alkyl radical but with 1 to not more than 18 C atoms.

The (C₁-C₈)-alkoxy radical corresponds to the (C₁-C₈)-alkyl radical with the proviso that the latter is linked via an oxygen atom to the molecule.

(C₂-C₈)-Alkoxyalkyl radicals mean those in which the alkyl chain is interrupted by at least one oxygen function, it not being possible for two oxygen atoms to be connected together. The number of carbon atoms indicates the total number of carbon atoms present in the radical.

A (C₃-C₅)-alkylene bridge is a carbon chain with three to five C atoms, and this chain is linked by two different C atoms to the molecule under consideration.

The radicals just described in the preceding sections may be substituted one or more times by halogens and/or hetero-atom-containing radicals having N, O, P, S, Si atoms. These are in particular alkyl radicals of the abovementioned type which have one or more of these heteroatoms in their chain and which are linked via one of these heteroatoms to the molecule.

(C₁-C₈)-Acyloxy means in the context of the invention a (C₁-C₈)-alkyl radical as defined above which has a maximum of 8 C atoms and which is linked via a COO function to the molecule.

(C₁-C₈)-Acyl means in the context of the invention a (C₁-C₈)-alkyl radical as defined above which has a maximum of 8 C atoms and which is linked via a CO function to the molecule.

A (C₆-C₁₈)-aryl radical means an aromatic radical having 6 to 18 C atoms. These include in particular compounds such as phenyl, naphthyl, anthryl, phenanthryl, biphenyl radicals or systems of the type described above which are fused to the relevant molecule, such as, for example, indenyl systems, which may optionally be substituted by (C₁-C₈)-alkyl, (C₁-C₈)-alkoxy, (C₂-C₈)-alkoxyalkyl, NH(C₁-C₈)-alkyl, N((C₁-C₈)-alkyl)₂, OH, O(C₁-C₈)-alkyl, NO₂, NH(C₁-C₈)-acyl, N((C₁-C₈)-acyl)₂, F, Cl, CF₃, (C₁-C₈)-acyl, (C₁-C₈)-acyloxy, (C₇-C₁₉)-aralkyl, (C₄-C₁₉)-heteroaralkyl.

A (C₇-C₁₉)-aralkyl radical is a (C₁-C₈)-alkyl radical linked via a (C₆-C₁₈)-aryl radical to the molecule.

A (C₃-C₁₈)-heteroaryl radical means in the context of the invention a five-, six- or seven-membered aromatic ring system composed of 3 to 18 C atoms which has heteroatoms such as, for example, nitrogen, oxygen or sulphur in the ring. Such heteroaromatic radicals are regarded in particular as being such as 1-, 2-, 3-furyl, such as 1-, 2-, 3-pyrrolyl, 1-, 2-, 3-thienyl, 2-, 3-, 4-pyridyl, 2-, 3-, 4-, 5-, 6-, 7-indolyl, 3-, 4-, 5-pyrazolyl, 2-, 4-, 5-imidazolyl, acridinyl, quinolinyl, phenanthridinyl, 2-, 4-, 5-, 6-pyrimidinyl. The heteroaromatic systems may be substituted in the same way as the abovementioned (C₆-C₁₈)-aryl radicals.

A (C₄-C₁₉)-heteroaralkyl means a heteroaromatic system corresponding to the (C₇-C₁₉)-aralkyl radical.

(C₃-C₈)-Cycloalkyl means cyclopropyl, cyclobutyl, cyclo-pentyl, cyclohexyl and cycloheptyl radicals, etc. These may be substituted by one or more halogens and/or N—, O—, P—, S—, Si atom-containing radicals and/or have N—, O—, P—, S atoms in the ring, such as, for example, 1-, 2-, 3-, 4-piperidyl, 1-, 2-, 3-pyrrolidinyl, 2-, 3-tetrahydrofuryl, 2-, 3-, 4-morpholinyl. The cycloalkyl radicals may be substituted in the same manner as the abovementioned (C₆-C₁₈)-aryl radicals.

A (C₃-C₈)-cycloalkyl-(C₁-C₈)-alkyl radical means a cycloalkyl radical as described above which is linked via an alkyl radical as indicated above to the molecule.

Halogens (Hal) are fluorine, chlorine, bromine, iodine. Hal′ are chlorine, bromine, iodine.

N-Acyl groups mean besides a (C₁-C₈)-acyl radical also a protective group which is generally customarily employed in amino acid chemistry to protect nitrogen atoms. As such, particular mention should be made of: formyl, acetyl, Moc, Eoc, phthalyl, Boc, Alloc, Z, Fmoc, etc.

The term enantiomerically enriched or enantiomeric excess means in the context of the invention the proportion of one enantiomer mixed with its optical antipode in a range from >50% and <100%. The ee is calculated as follows:

([enantiomer1]−[enantiomer2])/([enantiomer1]+[enantiomer2])×100=ee[%]

The specification of the enantiomerically enriched β-amino acids and their derivatives and oxazines includes in the context of the invention all possible diastereomers, the intention also being to name both the optical antipodes of each diastereomer.

1: A process for preparing enantiomerically enriched compounds selected from the group consisting of N-acylated β-amino acid esters, β-amino acid esters, N-acylated β-amino acids, β-amino acids and 4,5-dihydrooxazin-6-ones by kinetic racemate resolution of the 4,5-dihydrooxazin-6-one formed from a corresponding racemic N-acylated β-amino acid, wherein the 4,5-dihydrooxazin-6-one is reacted with catalytic amounts of an enantiomerically enriched compound of the general formula (Ia) or (Ib) 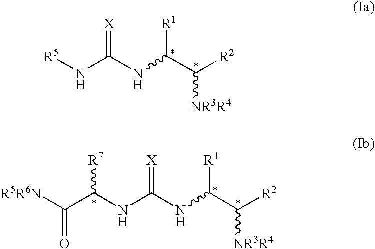
in which
* represent chiral centres,
X may be O or S or NHR¹ or NR¹R²,
R, R¹, R², R³, R⁴ are independently of one another (C₁-C₈)-alkyl, (C₁-C₉)-alkoxy, HO—(C₁-C₈)-alkyl, (C₂-C₈)-alkoxyalkyl, (C₆-C₁₈)-aryl, (C₇-C₁₉))-aralkyl, (C₃-C₁₈)-heteroaryl, (C₄-C₁₉)-heteroaralkyl, (C₁-C₈)-alkyl-(C₆-C₁₈)-aryl, (C₁-C₈)-alkyl-(C₃-C₁₈)-heteroaryl, (C₃-C₈)-cycloalkyl, (C₁-C₈)-alkyl-(C₃-C₈)-cycloalkyl, (C₃-C₈)cycloalkyl-(C₁-C₈)-alkyl, and
R¹ and R² and/or R² and R³ may be connected together via a (C₃-C₅)-alkylene bridge, in the presence of a nucleophile.

2: The process according to claim 1, wherein
racemic 4,5-dihydrooxazin-6-one of the general formula (II) 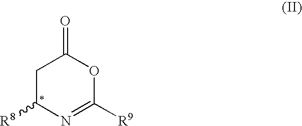
in which
* represent chiral centres,
R⁸, R⁹(C₁-C₁₈)-alkyl, (C₁-C₈)-alkoxy, HO—(C₁-C₈)-alkyl, (C₂-C₈)-alkoxyalkyl, (C₆-C₁₈)-aryl, (C₇-C₁₉)-aralkyl, (C₃-C₁₈)-heteroaryl, (C₄-C₁₉)-heteroaralkyl, (C₁-C₈)-alkyl-(C₆-C₁₈)-aryl, (C₁-C₈)-alkyl-(C₃-C₁₈)-heteroaryl, (C₃-C₈)-cycloalkyl, (C₁-C₈)-alkyl-(C₃-C₈)-cycloalkyl, (C₃-C₈)-cycloalkyl-(C₁-C₈)-alkyl,
is employed in the kinetic racemate resolution.

3: The process according to claim 1,
wherein
one or more alcohols selected from the group consisting of allyl alcohol, methanol, ethanol, phenol, n- and iso-propyl alcohol and n-, tert-, sec- and iso-butanol are employed as nucleophile for the reaction.

4: The process according to claim 1,
wherein
the catalysts of the general formulas (Ia) and (Ib) are employed in the range from 0.01 to 40 mol % based on the substrate.

5: The process according to claim 1,
wherein
a temperature of between 15° C. and 40° C. is set during the reaction.

6: The process according to claim 1,
wherein
the reaction is carried out in organic aprotic solvents.

7: The process according to claim 1,
wherein the
catalysts with an enantiomeric enrichment of >80% are employed.

8: The process according to claim 1, wherein the reaction is carried out in toluene.
